(19) United States
(12) Patent Application Publication
(10) Pub. No.: US 2025/0049589 A1
(43) Pub. Date: Feb. 13, 2025

(54) ORTHOPAEDIC DEVICE AND METHOD FOR PRODUCING SAME

(71) Applicant: OTTO BOCK HEALTHCARE PRODUCTS GMBH, Wien (AT)

(72) Inventor: Dirk SEIFERT, Wien (AT)

(21) Appl. No.: 18/718,997

(22) PCT Filed: Dec. 12, 2022
(86) PCT No.: PCT/EP2022/085368

§ 371 Date: Jun. 12, 2024

(30) Foreign Application Priority Data
Dec. 13, 2021 (DE) 10 2021 006 127.6

(51) Int. Cl. A61F 2/70; A61F 2/64; A61F 2/66

(52) U.S. Cl. CPC A61F 2/70; A61F 2/64; A61F 2/6607

(57) ABSTRACT

The invention relates to a method for controlling an orthopaedic device (100) for the lower extremity, having a top part (2) and a bottom part (3), which are fitted with one another in an articulated manner about at least one pivot axis (4) so as to form a joint (5), and having at least one actuator (6) which is coupled to a control device (7) which activates or deactivates the actuator (6) on the basis of sensor data from at least one sensor (8) coupled to the control device (7), in order to influence a pivoting resistance and/or a movement of the top part (2) relative to the bottom part (3), wherein an orientation and/or displacement of the orthopaedic device (100) in the frontal plane is detected using the sensor data and the actuator (6) is activated or deactivated or a target value for the actuator (6) is modulated on the basis of the orientation and/or displacement in the frontal plane.

The invention relates to a method for controlling an orthopedic device of the lower extremity having an upper part and a lower part, which are mounted with one another in an articulated manner around at least one pivot axis to form a joint and having at least one actuator, which is coupled to a control unit that activates or deactivates the actuator on the basis of sensor data of at least one sensor coupled with the control unit in order to influence a pivot resistance and/or a relative movement of the upper part in relation to the lower part, and such an orthopedic device, in particular for carrying out the method.

Orthopedic devices of the lower extremity are understood in particular as orthoses and prostheses. Orthoses are orthopedic aids which are applied to an existing limb and guide, restrict, or assist movement. Drives, actuators, and/or resistance units, which can be adjusted or set via an actuator, are arranged between components connected to one another in an articulated manner. The adjustment can take place on the basis of sensor data which are transmitted to a data processing unit. Orthoses are also understood in the scope of this application as exoskeletons which are applied to the body of a patient and form an external support structure, in particular to guide and influence the movements of a user, for example, to assist them by way of drives or brake them via resistance units. Orthoses and exoskeletons as the special cases thereof can also be used and employed, in addition to the assistance of the daily activities, for training purposes or for therapeutic purposes.

Prostheses replace limbs which are not present or are no longer present. The simplest prosthesis components have a solely cosmetic function or complete a limb, for example, in that a distal phalanx is replaced. In the course of time, prostheses have become more complex, and multiple prosthesis components have been arranged and fastened on one another and, for example, connected to one another via joints. Complex mechanical drive devices have been developed, for example, to move prosthetic hands or prosthetic feet. Hydraulic or other damper units or resistance units have been arranged on joints to modify the behavior of prosthesis components and prosthesis systems, in order to enable the most natural possible movement sequence. Drives have been integrated into prosthesis components to assist movements, so that active prostheses have resulted. Furthermore, sensors have been arranged on prosthesis components or on a prosthesis user in order to detect the current movement behavior or the current positions or settings of prosthesis components in relation to one another and to predict the future movement behavior and in order to change settings on resistance units and/or drives. Highly complex prosthesis systems having multiple prosthesis components arranged on one another have thus arisen, which have a large number of mechanical, electrical, and mechatronic components.

A prosthesis system of the lower extremity can in particular have a thigh socket, on the distal end of which a prosthetic knee joint, a prosthetic lower leg, and a prosthetic foot are fastened. Such a prosthesis system has, for example, two or more joints which can each be provided with resistance units and/or drives or actuators.

A method for controlling an artificial orthotic knee joint or prosthetic knee joint is known from EP 2 816 979 B1, in which the deflection resistance is changed on the basis of the detection of an absolute angle of a lower leg component. The determined absolute value of the lower leg component is compared to a threshold value, and if the threshold value is reached or exceeded, the deflection resistance is changed.

A method for controlling an orthopedic foot part having an ankle joint is known from EP 2 649 968 B1, in which torques occurring at the ankle joint, the ankle angle, and the absolute angle of a foot part in relation to the vertical are determined. The rolling of the foot in the standing phase, the positioning of the foot part in the swinging phase, and the positioning and mobility of the foot part during standing up are controlled by means of a damping arrangement as a function of the measured values.

Moreover, to switch between various operating modes, it is known that orthopedic devices of the lower extremity are especially loaded in order to set a special mode. A repeated rhythmic loading in a specific period of time in a specific loading direction is assessed as a switching signal in order to then activate special programs, for example, for going up stairs.

Intentionally switching the operating modes requires a high level of attentiveness by the user of the orthopedic device.

The object of the present invention is to provide a method and an orthopedic device, using which users of orthopedic devices of the lower extremity can carry out activities of daily life more easily and using which it is possible to carry out a more versatile use of the orthopedic device.

This object is achieved according to the invention by a method having the features of the main claim and an orthopedic device having the features of the other independent claim. Advantageous embodiments and refinements of the invention are disclosed in the dependent claims, the description, and the figures.

The method for controlling an orthopedic device of the lower extremity having an upper part and a lower part, which are mounted with one another in an articulated manner around at least one pivot axis to form a joint, and having at least one actuator, which is coupled to a control unit that activates or deactivates the actuator on the basis of sensor data of at least one sensor coupled to the control unit, in order to influence a pivot resistance and/or a relative movement of the upper part in relation to the lower part, provides that via the sensor data an orientation, displacement, or a combination of orientation and/or displacement of the orthopedic device in the frontal plane is detected and that on the basis of the orientation and/or displacement in the frontal plane, the actuator is activated or deactivated and/or a target value for the actuator is modulated. While exclusively the movements in the sagittal plane are analyzed, evaluated, and used to modify the resistances or drives in the prior art, according to the invention a lateral inclination, a lateral rotation, and/or a lateral movement in the frontal plane of a user is detected and used to control resistances and/or drives, in particular to control movement resistances and/or movements. The incorporation of the sensor data with respect to the orientation and/or displacement of the orthopedic device within the frontal plane, in general transversely to the typical forward movement, can also be used in addition to other control methods. In this case, orientations and/or displacements within the frontal plane of individual components of the orthopedic device or also of multiple or all components of the orthopedic device are detected. In particular in multi-joint devices such as orthoses or prostheses, which comprise both the ankle joint and the knee joint, the joint axes of the orthopedic devices are regularly oriented parallel or approximately parallel to one another and are located in the frontal plane when the joint axes stand one over another. The articulation between an upper part and a lower part can also be formed by multiple axes. Such joints are referred to as polycentric joints. Orthopedic devices can also span the hip joint. Such a hip joint can be designed so that it may also be pivoted around an axis which essentially lies in the frontal plane. However, it is also possible that a pivot around further axes is possible, for example, an abduction movement around an axis normal to the frontal plane. In addition to the pivoting around axes, an additional or alternative displaceability and/or a spherical mounting is advantageous in particular for the hip joint. In general, the joint axes are oriented horizontally in the frontal plane in the starting position here. In the starting position the upper parts and lower parts are in an essentially perpendicular orientation, so that the longitudinal extension of the orthopedic device essentially corresponds to the vertical. As soon as the orthopedic device is pivoted in the frontal plane, the orientation of the longitudinal extension changes and therefore the orientation of the pivot axes also changes. The pivoting takes place here in the standing phase, with a foot planted, around a pivot point or a pivot axis at the distal end or in the distal end area of the orthopedic device. The foot or the distal end remains stationary relative to the underlying surface, the body of the user of the orthopedic device is moved. If, for example, the right leg is treated using the orthopedic device, a pivot in the frontal plane in the standing place predominantly takes place in that the left leg is moved away from the orthopedic device to the left. A translation of the left foot to the right side of the treated leg takes place rather rarely, wherein this is also a movement in the frontal plane. A pivot and/or movement in the frontal plane can also be combined with a pivot, rotation, and/or movement in another plane, for example, if the leg is pivoted diagonally forward via a distal rolling point. The movement thus partially takes place in the frontal plane, but not necessarily exclusively. The control can relate to a movement component, in particular a movement component in the frontal plane.

In the swinging phase of the treated right side, for example, the untreated leg, for example, remains on the ground and the treated right leg is raised and spread out to the right, for example, to displace the entire body to the right. A translation to the left side of the left untreated leg is also possible here and results in a pivot or displacement in the frontal plane. Such movements or states in the frontal plane are detected and used as a basis for determining whether an actuator is activated or deactivated or a target value for the actuator is modulated or changed, for example, to influence a pivot resistance of an upper part in relation to a lower part via a resistance unit and/or relative displacement of upper part in relation to lower part via a drive, for example, a motor, or a release of stored energy. Multiple actuators can also be actuated. If no movement takes place in the frontal plane, but rather exclusively, for example, in the sagittal plane, the actuation of the at least one actuator changes and other control mechanisms engage and/or another control program is activated, in order to influence the pivot resistance and/or a relative movement of the upper part in relation to the lower part via the actuator. The method according to the invention is in particular a part of a complex controller of the orthopedic device, via which resistances to a pivot are influenced or a drive is activated or deactivated on the basis of a large amount of sensor data or a target value for the actuator is modulated.

In a refinement of the method, multiple degrees of freedom of the orthopedic device are controlled. This can be one or more additional degrees of freedom of a joint in this case. Alternatively or additionally, multiple joints and/or components can also be controlled. An orthopedic device can encompass, for example, ankle and knee, knee and hip, or ankle, knee, and hip. A joint can moreover have multiple degrees of freedom, for example, a hip joint, which permits both flexion-extension and also adduction-abduction. However, all degrees of freedom of an orthopedic device do not necessarily have to be controlled.

In a refinement of the method, it is provided that during the use of the orthopedic device in the applied state, the sensor data are determined and that the actuator is activated or deactivated or a target value for the actuator is modulated in order to influence the pivot resistance and/or a relative movement of the upper part in relation to the lower part.

The orientation and/or the displacement of the orthopedic device or parts of the orthopedic device is detected and determined in one embodiment via a spatial position sensor, at least one IMU (inertial measurement unit), and/or at least one angle sensor. The angle sensors or the angle sensor detect(s) the position of upper part in relation to lower part, individual components in relation to one another, or also of the components relative to a body part or another reference element on the user and enable(s) the determination of the orientation of the entire orthopedic device, multiple parts thereof, or also only a part thereof within the respective plane, in particular within the frontal plane. The orientations of components or the entire orthopedic device and, if necessary, the orientation of the components in relation to one another can be determined directly via a spatial position sensor or an IMU and, after evaluation of the controller, corresponding commands can be transmitted for activating, deactivating, or modulating a target value of the actuator to change the pivot resistance and/or the relative movement. A relative angle between two components can be determined from the respective absolute angles, for example, by two IMUs.

In a refinement, forces, torques, and/or accelerations of the entire orthopedic device and/or its components are detected via sensors and used as the basis for the control. In conjunction with the detection of pivots and/or displacements within the frontal plane, additional parameters are used for the control of the actuator. A disappearance of axial forces shows, for example, that the orthopedic device is in a swinging phase. Different force distributions or introductions of torques around pivot axes enable a determination of movements, movement changes, states, and probable future movements or loads, so that on the basis of the forces, torques, and/or accelerations, in particular in conjunction with data on the orientation and/or displacement within the frontal plane, the actuator is supplied with corresponding commands. Forces and torques can be determined via a deformation, shift, tilt, and/or a combination thereof. For example, an acting force and/or a torque can be concluded from the deformation of an elastic or compressible body and/or a tilt or shift resulting therefrom. A force and/or a torque can also be concluded via the rate of shift or deformation of a damper or viscous element.

In one embodiment, only individual measured variables in the frontal plane are determined or measured and a pivot in the frontal plane is thus concluded. For example, a torque around the ankle can only be determined in the sagittal plane, while the acceleration is determined both in the sagittal plane and in the frontal plane. It is also possible that individual measured variables are exclusively determined in the frontal plane. An orthopedic device can be produced cost-effectively and robustness can be increased by way of a minimal sensor set.

In one embodiment, the orthopedic device is designed as a prosthesis or orthosis and has an artificial knee joint and/or ankle joint, wherein an actuator is assigned to each joint. If both a knee joint and an ankle joint are present, two actuators can be provided in order to change a pivot resistance around the respective joint individually and independently of one another or introduce or influence a relative movement between the respective upper part and the respective lower part. There is also the possibility that only a single actuator is assigned to two joints, via which a corresponding increase or reduction of a pivot resistance is achieved or a displacement of upper part in relation to lower part is caused. More than two joints and one or more actuators can also be arranged and controlled.

In one refinement, translational displacements of the orthopedic device are detected and also used as the basis for the control. The detection and calculation of translational movements of an orthopedic component can be carried out, for example, via a distance integration in the respective plane and permits conclusions about the type and the scope of the movement of the orthopedic components at the respective reference point, for example, in the distal end area of the orthopedic device or at a joint.

In one refinement, relative distances between components of the orthopedic device and/or between the orthopedic device, limbs, and/or body parts of the person using it, the surroundings, and/or other orthopedic devices are determined. Distances to the surroundings, in particular to the ground, can also be determined. Relative distances can be determined along a kinematic chain, for example, by one or more relative angle and/or absolute angle sensors with the aid of segment lengths. For example, if lower leg length and thigh length are known, the relative distance of ankle and hip can be calculated from the knee angle. Alternatively or additionally, distances can be directly measured. In one embodiment, at least one distance is determined between two points via a surroundings sensor system. The surroundings sensor system determines the arrangement of objects in relation to the orthopedic device and/or between objects, for example, by interaction with electromagnetic radiation. Objects can be detected, for example, by the electromagnetic radiation emitted, reflected, or reemitted by them. It is possible to detect objects by way of one or more cameras which operate in the visible and nonvisible wavelength range. Depth image cameras can also be used. Lidar, sonar, radar, and/or similar sensor systems can be used in order to detect the surroundings. Object distances can be determined, inter alia, by time-of-flight measurements and/or triangulation. Relative speeds can be determined, for example, via the Doppler effect. Multiple sensors and/or sensor arrays can be arranged on the orthopedic device in order to determine the direction of an object and/or the arrangement, location, and/or orientation of objects in relation to the orthopedic device from multiple measurements, for example, multiple distance and/or time-of-flight measurements. The one or more objects or the surroundings can include other components of the orthopedic device, limbs, or body parts of the person's own body, the underlying surface, and/or objects in the surroundings of the person using it. It is also possible to detect the geometric configuration by way of external sensors, for example, one or more cameras, and pass on the information to the controller of the orthopedic device. Distances and/or geometric arrangements can also be determined via one or more receivers and transmitters which are located at different positions. Distances and arrangements of components can in particular be determined in the frontal plane and/or calculated or detected in the frontal plane.

In one embodiment of the method, in the standing phase, in the event of an inclination or pivot of the treated side having the orthopedic device in the medial direction, a reduction of a flexion resistance or flexion movement is initiated in dependence on the inclination, in particular upon reaching a threshold value of the inclination. During the standing phase, an inclination or pivot in the medial direction takes place by way of a pivot or tilt around a distal support point or a distal pivot axis in the direction toward the body center relative to the longitudinal extension of the orthopedic device in the starting position or relative to the vertical or the line of gravity. Depending on the pivot, in one embodiment the flexion resistance is reduced or, in the case of an active drive, a flexion torque is applied and/or a flexion movement is initiated, in particular if the inclination or the pivot angle during a movement of the treated side in the standing phase exceeds a threshold value. For a knee joint, this means that a flexion is facilitated or initiated while still in the standing phase, so that in particular in orthoses, the knee is bent and thus the foot can be raised or is raised more easily. Moving sideways and/or a direction change is thus also facilitated with prostheses. It is also possible in the case of a pivot to the lateral to adapt resistances and/or movements, for example, in a similar manner as in the case of a pivot to the medial.

In one embodiment, upon a reduction of the axial load or in a raising phase of the treated side, a reduction of the flexion resistance or a flexion is initiated. By way of a reduction of the axial load or by way of the detection of a raising phase, which can take place after identification or detection of the terminal standing phase by evaluation of sensor data such as force distribution, force curve, torque distribution, torque curve, and angle positions of components in relation to one another, the flexion resistance is already reduced or a flexion is initiated as a precaution. This is advantageous in particular with artificial knee joints such as prosthetic knee joints and orthotic knee joints. In knee joints, the flexion resistance is reduced or a flexion is actively initiated, and, in particular in orthotic ankle joints, for example, the dorsiflexion resistance is reduced or a dorsal reflection is initiated, in particular in the swinging phase, in each case to facilitate swinging through and provide a greater ground clearance.

In one embodiment, a push off is generated in an ankle joint upon a pivot in the frontal plane in the standing phase, in which a pulse upward is provided by a plantar flexion and/or an excessively fast sinking of the body center of gravity is prevented. The push off can, inter alia, be chronologically controlled or can be the consequence of a force-displacement characteristic, for example, an elastic behavior. The intensity of the push off, for example, the duration, the extent of movement, the maximum force, the power, and/or the work performed, can be adapted with the pivot in the frontal plane, for example, the pivot in the terminal standing phase, in particular reduced or increased during a pivot.

One embodiment provides that a reduction of a flexion resistance or an active flexion is initiated only upon a forward inclination or forward pivot or up to a certain extent of backward inclination or backward pivot of the orthopedic device or a component thereof. A forward pivot is a pivot within the sagittal plane to the anterior, which is typically the walking direction of a user of an orthopedic device and typically corresponds to the direction of view.

Upon an inclination or pivot of the treated side in the standing phase in the medial direction with simultaneous detection of a backward inclination and/or a backward pivot and/or upon detection of a sufficiently small forward inclination and/or forward pivot, in one embodiment, no reduction or an increase of a flexion resistance takes place. Alternatively or additionally, a stretching torque can also be applied. The orthopedic device thus becomes more stable overall and a greater degree of security is provided for the user of the orthopedic device.

Upon the inclination or pivot of the treated side in the standing phase in the medial direction with simultaneous detection of a backward inclination or backward movement or backward pivot and/or upon detection of a sufficiently small forward inclination and/or forward pivot, a flexion movement is stopped or an extension movement is initiated, so that by corresponding activation or deactivation of a drive or change of a target value for the actuator, a relative movement between the upper part and the lower part around the pivot axis is changed. This is expedient in particular in knee joints to achieve better standing security.

The maximum pivot angle of the upper part in relation to the lower part can be adjusted depending on the inclination or pivot of the orthopedic device in the frontal plane. The greater the pivot of the entire orthopedic device or also only a component thereof is within the frontal plane, the greater the pivot angle of the upper part to the lower part can be set, so that, for example, a greater knee flexion is enabled. The change in the maximum pivot angles can take place in steps or continuously.

Upon an inclination or pivot of the side treated with the orthopedic device in the standing phase in the medial direction, an artificial ankle joint can be driven in the plantar flexion direction or the dorsiflexion resistance can be increased or not reduced, in order either to execute a plantar flexion or to enable or assist pressing down to the side.

If, in the standing phase upon an inclination or pivot of the treated side, a flexion torque is established simultaneously, for example, a torque which causes or would cause a knee flexion or a dorsiflexion within the ankle joint, it is provided in one embodiment that no reduction or an increase of a flexion resistance is initiated or an extension torque is applied by a drive. This increases the stability of the orthopedic device and facilitates walking around curves, in particular tight curves.

Upon the pivot of the treated side in a raising phase of the swinging phase in the lateral direction, when the treated side is moved away from the body, in one embodiment a reduction of a flexion resistance takes place, in particular against a knee flexion. Alternatively or additionally, an active knee flexion is initiated. The dorsal extension resistance of a foot or foot part can be reduced and/or a dorsal extension can be initiated.

An extension in an artificial knee joint can be delayed or prevented in the swinging phase, in particular upon a pivot of the treated side in the lateral direction, in order to carry out a complete movement up to the end, in particular to enable an appropriate ground clearance.

An extension can be released or caused in a time-controlled manner in an artificial knee joint. Alternatively or additionally, depending on the pivot angle and/or the movement, in particular after reaching a threshold value, a change of the pivot speed, a change of the pivot direction, and/or on the basis of a hip extension movement and/or the thigh movement, the extension in the artificial knee joint can be released or caused.

In a hip-encompassing orthopedic device, a hip extension can initially be prevented or slowed and released at a later time. A release can also be a reduction of the slowing.

A special mode of the control can be activated or deactivated on the basis of the inclination and/or displacement in the frontal plane.

In one embodiment, a movement mode and/or a special function is interrupted, changed, or departed when a lateral movement and/or a pivot in the frontal plane is identified. A movement mode can be a control which controls a specific movement sequence, for example, walking up a staircase, sitting down, or running. An orthopedic device can change fluidly from one movement mode into another, for example, from standing up into walking forward. For many movement sequences, a lateral movement and/or a pivot in the frontal plane is/are atypical and can be used as an indicator that a change is being made from the original movement sequence into another movement sequence and thus the control or the underlying control law is to be adapted. If, for example, during a rapid hip flexion, which is initially viewed as a movement sequence of walking up stairs, and the orthopedic device provides a corresponding control for this movement sequence, an additional lateral movement and/or a pivot in the frontal plane is identified, for example, an abduction of the hips, the movement sequence of walking up the stairs is preferably terminated. In the further course of things, for example, a switch is made to a base state, which carries out a partial knee extension instead of an active knee flexion. The behavior of the orthopedic device is adapted so that upon a pivot in the frontal plane, the control for a specific movement sequence is terminated and the behavior changes. In some movement sequences, a pivot in the frontal plane can also be an indicator of a safety-critical situation, which makes it necessary to change the control strategy. A pivot in the frontal plane, in particular around a distal pivot axis or a pivot point, can indicate, for example, a loss of equilibrium, if the actual movement mode is a movement which essentially takes place in the sagittal plane. As a result, the control can be adapted. An adaptation of the control can be, for example, a change of the movement resistance, a force-distance characteristic, and/or the adaptation of a setting and/or movement. It is possible that a movement mode is only temporarily interrupted due to a pivot in the frontal plane, for example, as long as a lateral inclination is present. If a lateral inclination is no longer present, the corresponding movement mode and the control connected thereto are executed again.

A special function can be a function which is set by a special user interaction. A function for riding bicycles, a sport mode, a special function for driving cars, and a standby function are mentioned as examples. A special function can be activated by a specific movement and/or load pattern, for example, a rocking pattern on the forefoot. Alternatively or additionally, it is possible to switch into a special function via an interface, for example, an operating element or an app. Moreover, it is possible that a special function is ended, inter alia, when a pivot in the frontal plane is recognized. For example, a bicycle function can be departed when a lateral movement and/or lateral pivot is recognized, in particular a sudden lateral movement of the foot and/or spreading out of the leg. Such a movement is an indicator that the person wishes to step down from the bicycle. It is therefore advantageous to end the bicycle function and the control connected thereto and to change into a mode which is particularly suitable for standing.

If the orthopedic device is controlled on the basis of a pivot in the frontal plane, it is possible that a certain minimum degree of movement or change of the movement is necessary with time and its arbitrary derivatives in order to have an influence on the control. This can be implemented via one or more threshold values and/or via more complex algorithms, for example, via a majority decision via multiple values or via artificial intelligence.

All control algorithms which have a pivot in the frontal plane or variables derived therefrom as the input variable can also have further input variables, in particular movements in other directions, loads, and/or items of information from other sensors, which influence the behavior of the orthopedic device.

In one embodiment, the control is combined on the basis of the pivot in the frontal plane via at least one biosignal. Biosignals are any signals which contain items of information about properties of the body and information flows in the body. A biosignal can be an electrical signal, such as nerve signals, electrical signals upon contraction of the musculature such as EMG or electromyography, but also brain waves. If items of information are coded in a signal, decoding can initially take place before the signal is used for the control. A biosignal can be a chemical or electrochemical signal, for example, the concentration of a substance, the interaction of molecules, or an electrochemical gradient. Biosignals can also be conductivities of tissues and/or body parts. Biosignals can also be mechanical parameters such as force, pressure, and length and the changes over time thereof, for example, the pulse or length change of a muscle. Biosignals can be detected invasively or noninvasively. In one embodiment, the control is modulated via at least the detected pivot in the frontal plane by at least one biosignal, in particular via a biosignal which is connected to a physical or intended muscle tension, contraction, and/or actuation of the movement apparatus. Due to the modulation by means of such a biosignal it is possible for the person wearing the device to influence the control and thus the movement and/or the movement resistance. The modulation can be a change between different control characteristics or the continuous change of the control characteristic, in particular of a movement resistance and/or a movement. The treated person can thus, for example, by tensing the musculature in a raising phase of a lateral step, determine the extent of knee flexion. With a passive degree of freedom, it is possible to control its movement resistance depending on the muscle tension, for example, to increase the movement resistance upon tensing. For example, the knee angle and/or the hip angle can be kept constant after a flexion phase by tensing the musculature and/or another biosignal and stretching with gravity can be prevented. Alternatively, the stretching movement can be slowed. As the muscle contraction dissipates, the movement is permitted again or the stretching resistance is reduced. Alternatively or additionally, a pivot in the frontal plane can have an influence on the signal processing of a biosignal. For example, a biosignal can have a different influence on the control of the orthopedic device depending on the lateral movement and/or inclination. It is not precluded that the orthopedic device is primarily controlled by biosignals.

In one embodiment, in a lowering phase of a lateral step and/or a pivot in the frontal plane, a plantar flexion of the foot is carried out, preferably in combination with a knee and hip extension. In the lowering phase, the step length is essentially already reached and the foot is guided to the ground. The control of the foot can be designed here such that the initial contact takes place with the forefoot. This can be achieved, for example, by a plantar flexion. The plantar flexion movement can be stopped as soon as the ground contact is achieved. It is possible that the plantar flexion is controlled depending on the thigh angle, the hip angle, and/or the knee angle. Alternatively or additionally, the plantar flexion can be controlled depending on the lateral inclination and/or movement, in particular the extent. The plantar flexion can also result from a force-shift characteristic, wherein this characteristic can also be varied on the basis of other measured variables, for example, a neutral point, a position which the foot would assume free of force, is changed. A neutral point is preferably shifted in the placement phase from a neutral foot position toward a plantar flexed position. By means of a plantar flexion movement in the placement phase, it is possible that the ground contact is established early. The plantar flexion can be controlled so that the body center of gravity has to be lowered less or not at all during a lateral step in order to establish the ground contact after a swinging phase. With a lateral inclination and neutral foot position, the functional vertical leg length shortens in relation to the extended vertical standing. This length difference can be partially or completely compensated by a plantar flexion, in particular in a placement phase. During the loading of the foot, preferably a dorsal extension is permitted against a movement resistance, in particular so that the sole of the foot rests completely on the ground at latest upon complete load transfer. The movement resistance can be comparatively small in relation to standing upon initial contact. The movement resistance can moreover be increased with the load, but also with decreasing pivot in the frontal plane. If the foot is moved upward or downward in the course of a lateral step, for example, to overcome a height difference, this can be compensated by a corresponding control of the foot.

In one embodiment, during a pivot in the frontal plane, knee, hip, and/or foot are at least partially controlled via the orientation of the lower leg, thigh, and/or the hip angle in the sagittal plane, in particular in the swinging phase, but also in a loading and/or unloading phase. This can involve a functional kinematic coupling of the degrees of freedom. Other parameters can also be incorporated in this coupling, for example, the lateral inclination and/or movement and the time sequence thereof. By way of a coordinated control it is possible to position the foot precisely and control it, for example, via the thigh movement. In one possible embodiment, the functional coupling is executed such that the foot remains below the hip. Alternatively, the foot can be left in its anterior-posterior position in relation to the hip. If the foot is located in front of the body upon lifting, for example, it can also be held in front of the body during the swinging phase of a lateral step. The functional coupling can be dependent on the pivot in the frontal plane.

A movement resistance is to be understood in particular as a reaction force which is necessary to hold a certain position and/or execute a movement. This is also true of a reaction torque during a pivot. The reaction force or the reaction torque can thus be dependent, in addition to a possible constant component, among other things, on the position and/or its derivatives. In the case of a block, no movement is permitted, due to which the reaction force corresponds to the applied force. This is also a movement resistance here. A movement resistance can be, for example, a speed-proportional damping, a linear or nonlinear elasticity, a constant force, or a superposition of these behaviors, to mention only a few. A movement resistance can be implemented by a mechanism having corresponding intrinsic properties, but also by control or regulation of a passive or active actuator. A combination of multiple passive and/or active actuators is also possible. Multiple components can also be arranged in series and/or in parallel to one another.

An active actuator is capable of performing work on its surroundings and is generally supplied with electrical or chemical energy. It is also possible that an actuator absorbs potential energy and emits it at a later time. Actuators can be implemented via electromechanical drives, such as motors or piezoelectric elements. Actuators can be designed as thermal actuators or as elements which contract, expand, and/or change their shape under the influence of an electromagnetic field or a heat flow. In the orthopedic device, rotational drives can be translated into linear movements and vice versa by mechanisms. Actuators can comprise hydraulic or pneumatic components. Actuators can also be driven by chemical processes, such as internal combustion engines or muscle fibers, in which a length change is induced by a chemical bond. Movement resistances can be designed, inter alia, as friction brakes, clamping mechanisms, hydraulic and/or pneumatic dampers having Newtonian and/or non-Newtonian fluids, magnetorheological dampers or brakes, magnetic powder brakes, hysteresis and/or eddy current brakes, linear and nonlinear springs, inertial masses, or other braking mechanisms. The movement resistance can be changed in terms of a block and/or unlocking via stops, locking and/or unlocking mechanisms, in particular with form fit. The orthopedic device can also have at least one stimulator. In particular, the at least one actuator can be designed as a stimulator and can change a movement resistance and/or control a movement, for example, by the electrical stimulation of a muscle which results in its tensing. A muscle can be directly stimulated. Alternatively or additionally, nerves innervating the muscles and/or parts of the central and/or peripheral nervous system can be stimulated. A stimulation can take place invasively and/or noninvasively. A stimulation can be carried out above all by electrical signals, but, among other things, also mechanically, thermally, and/or chemically. One example is the functional electrostimulation of muscles.

An actuator can be regulated so that a position, an angle, and/or a target speed is specified. Time curves of the target variables can also be specified. It is also possible to regulate an actuator so that a torque and/or a force and/or a corresponding course are specified. Alternatively or additionally, an actuator can be controlled so that it follows a force-displacement law, i.e., a force and/or a torque is generated depending on a displacement and/or rotation and the chronological changes thereof, and vice versa. The behavior of a spring or a damper can be simulated by means of such a control, for example.

In one embodiment, during a lateral movement and/or lateral pivot, at least one hip angle, one thigh angle, one knee angle, and/or one lower leg angle in the sagittal plane and/or the time curve thereof is used for the control.

A pivot can be a rotation, a translation, or a combination of rotation and translation. A pivot can be determined between two or more components or in relation to a reference system, such as an inertial system. A pivot can be a specific position, a change of the position over time, or also a time curve of locations and/or location changes. A lateral movement can also be a rotation, a translation, or a combination of rotation and translation.

The control of resistances and/or movements can also be carried out on the basis of complex criteria or calculation rules, for example, by means of methods of artificial intelligence. The adaptation of control variables can take place continuously, for example, as a continuous function of sensor variables, in discrete steps, and/or at discrete points in time or events. Algorithms for the control and criteria for the control can be self-learning and/or auto-adaptive.

Both a pivot in the frontal plane and pivots in other directions and planes can be controlled on the basis of, among other things, a pivot in the frontal plane.

The controls described for the orthopedic device upon pivoting in the medial direction can also be applied upon pivoting in the lateral direction and vice versa. It is also possible upon pivoting in the medial direction to actuate the orthopedic device differently than upon pivoting in the lateral direction and vice versa. The control can change continuously with the extent of the pivot, in particular, in the case of movement sequences which only take place partially in the frontal plane, to adapt the control continuously in relation to the control of a movement which does not take place in the frontal plane, in particular to control movement resistances and/or movements. In one embodiment, the control of a knee joint in the swinging phase is changed starting from a control for walking straight ahead with increasing pivot in the frontal plane, for example, the pivot in the terminal standing phase, and adapted to a swinging phase control for walking laterally. A successive adaptation can also take place during the rolling off in the standing phase. Alternatively, an adaptation can take place in multiple discrete steps.

In one embodiment, a pivot in the frontal plane is identified and one or more parameters connected thereto are stored for evaluation at a later time and/or for transfer to another component. For example, the number of lateral steps and/or their step length are detected, transmitted to an activity tracker, and displayed there.

In one embodiment, the control comprises at least one auto-adaptive or self-learning algorithm, which adapts at least one parameter of the control, wherein the adaptation takes place on the basis of a detected pivot in the frontal plane and/or the at least one parameter influences the control upon a pivot in the frontal plane.

The orthopedic device of the lower extremity having an upper part and a lower part, which are mounted with one another in an articulated manner around at least one pivot axis to form a joint, and at least one actuator, which is coupled with a control unit, which activates or deactivates the actuator on the basis of sensor data of at least one sensor coupled with the control unit, in order to influence a pivot resistance or a relative movement of the upper part in relation to the lower part, provides that the at least one sensor is designed and configured to detect sensor data about an orientation and/or displacement of the orthopedic device in the frontal plane and that the control unit is configured to activate or deactivate the actuator or modulate a target value for the actuator on the basis of the orientation and/or displacement in the frontal plane. Via the actuator, the upper part is moved relative to the lower part, a movement is blocked, or a movement between the upper part and the lower part is moderated or modulated. This is performed by introducing energy into the system. If, for example, an artificial knee joint is flexed as an orthopedic device, the functional leg length shortens. Vice versa, upon extension or stretching of the artificial knee joint, the functional leg length is increased. Upon the recognition of a lateral inclination within the frontal plane, both the standing leg and the swinging leg can be the subject of the inclination recognition. If the untreated leg is the standing leg, an inclination in the frontal plane at the beginning of a lateral step results in a shortening of the overall leg length, for example, due to active flexion of an artificial knee joint or a reduction of the flexion resistance. If the treated leg is then put down again, the change of the position in the frontal plane is detected and the leg length is increased and/or the flexion resistance is elevated while reducing an extension resistance.

The at least one sensor is designed in one embodiment as an IMU and is fastened on the upper part or the lower part. In particular, the orthopedic device is only equipped with one IMU or exclusively with IMUs as sensors for detecting items of information with respect to the orientation in the frontal plane and in other spatial planes. Via the IMU it is possible to detect or calculate positions, orientations, and accelerations of the upper part and/or the lower part. Upon the use of two IMUs, one of which is assigned to the lower part and the other to the upper part, the angle between the upper part and the lower part can be calculated from the two absolute angles or spatial position angles in the respective planes. In principle, a combination with other sensors is also possible, which are arranged on the orthopedic device and/or the user of the orthopedic device and determine corresponding sensor data. Criteria or calculated variables can be determined from the sensor data independently of the structure of the sensors, for example, a projection in a plane, a force introduction point, or the like which are used for activation or deactivation of the actuator or for a change of a target value for the actuator.

In one refinement, at least one force sensor, acceleration sensor, angle sensor, and/or torque sensor is arranged on the upper part and/or the lower part. A force sensor can be designed, for example, for detecting a ground contact. A compressible element, a deformable or displaceable element, or also an elastically mounted element can act on a force sensor or a contact switch used as a force sensor in order to detect, for example, whether the respective leg is in a standing phase or in a swinging phase.

The actuator can also act on a locking element, which causes mechanical formfitting locking or blocking of the upper part relative to the lower part, so that a maximum resistance to a pivot is provided. The formfitting locking can be direction-dependent, for example, as with a reversible ratchet, so that, for example, an extension is always possible even if a fiction is blocked, or vice versa.

Exemplary embodiments of the invention are explained in more detail hereinafter on the basis of the figures. In the figures:

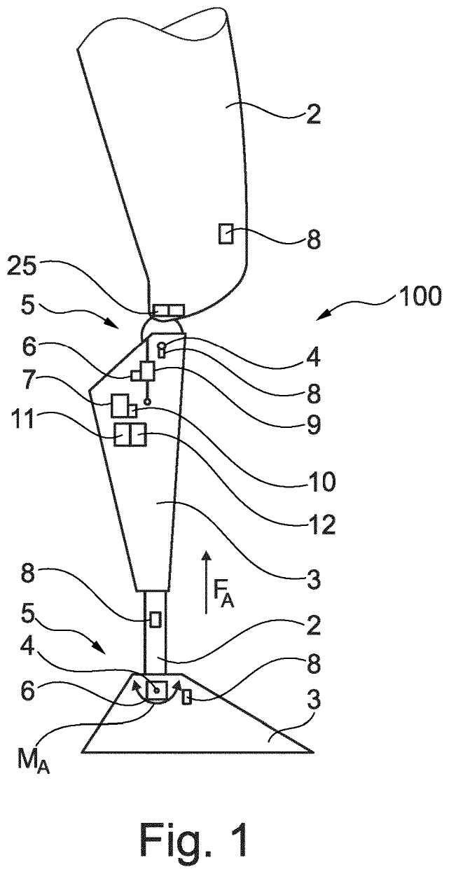

FIG. 1—shows a schematic representation of a prosthetic leg;

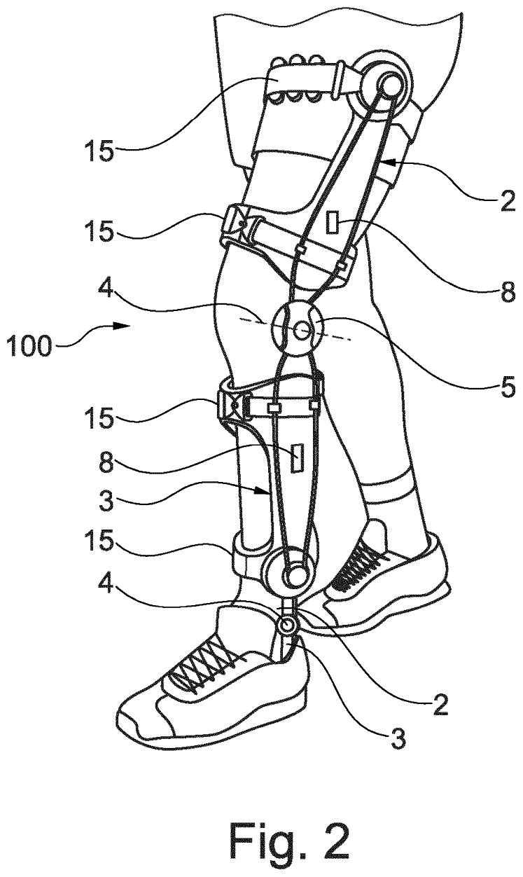

FIG. 2—shows a schematic representation of a KAFO;

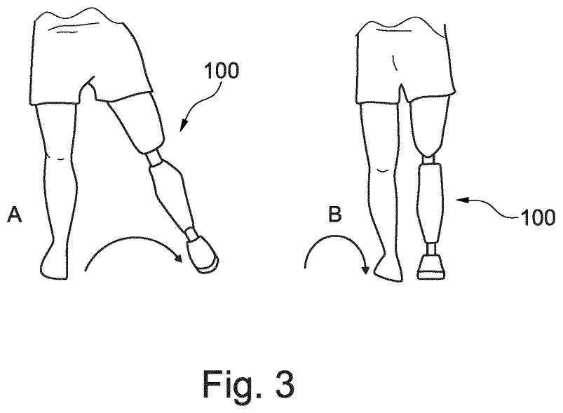

FIG. 3—shows a first movement sequence;

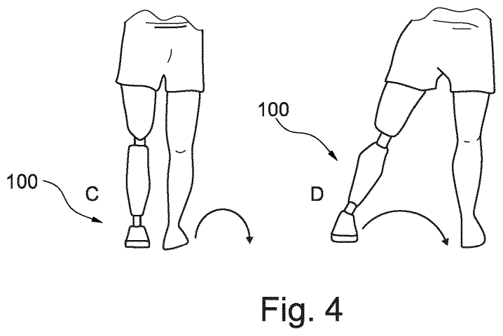

FIG. 4—shows a second movement sequence;

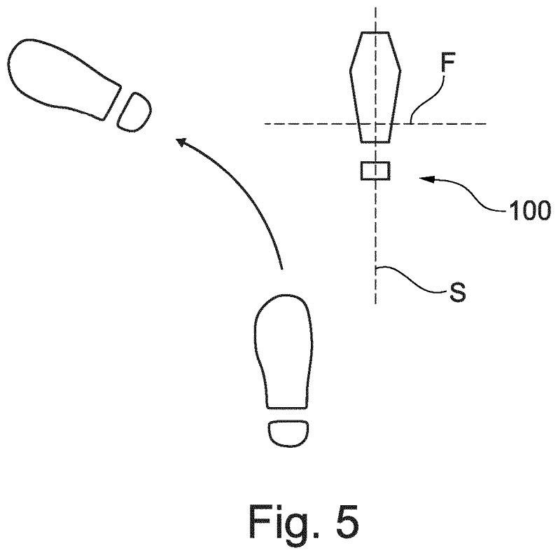

FIG. 5—shows a movement sequence for walking around the curve;

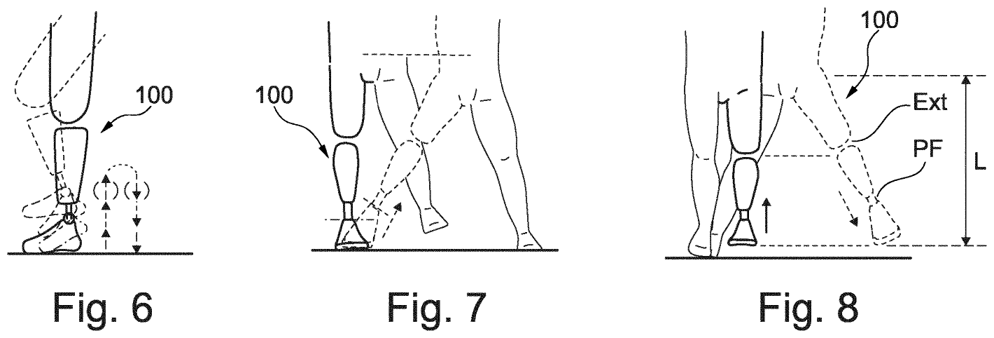

FIG. 6—shows the raising of a treated leg;

FIG. 7—shows a lateral step with an untreated leg;

FIG. 8—shows a lateral step with a treated leg;

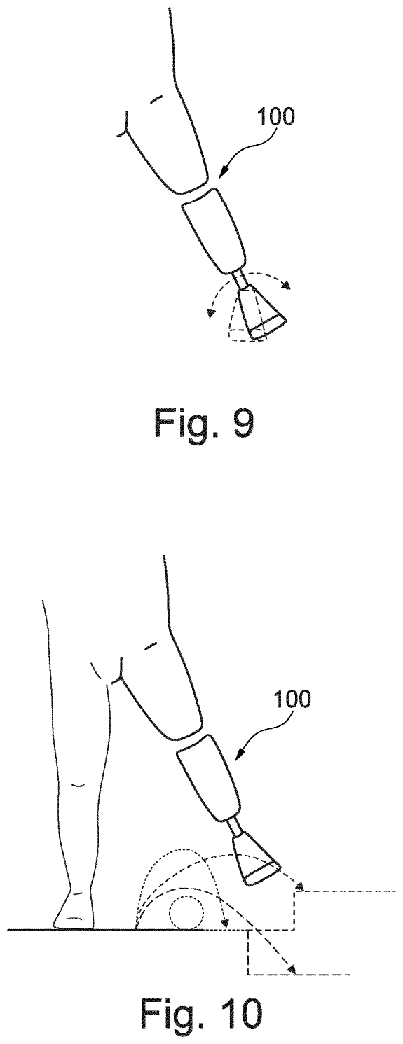

FIG. 9—shows a foot movement during a lateral step;

FIG. 10—shows laterally stepping over an obstacle; and

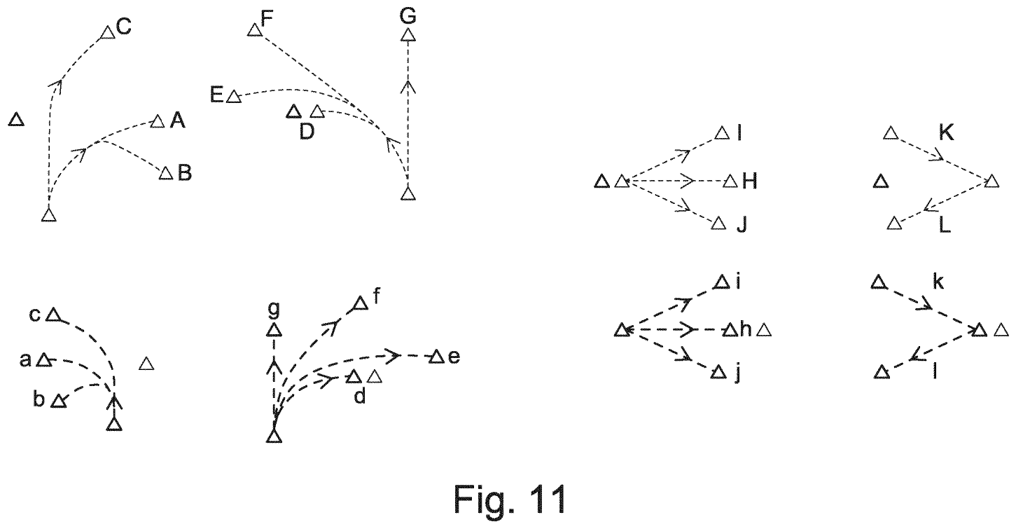

FIG. 11—shows different movement sequences.

FIG. 1 shows a schematic representation of an orthopedic device 100 in the form of a prosthetic leg having a first upper part 2 in the form of a thigh socket and a first lower part 3 in the form of a lower part of a prosthetic knee joint 5. The upper part 2 is mounted pivotably around a pivot axis 4 in relation to the prosthetic lower part 3. Fastening devices 25 for securing the thigh socket on the prosthetic knee joint 5 are arranged or formed on the upper part 2. The fastening devices 25 are, for example, a pyramid adapter with a corresponding receptacle. The first lower part 3 in the form of a lower leg part has a lower leg tube at its distal end, which is in turn used as a second upper part 2 for an articulated connection to a prosthetic foot as a second lower part 3. The prosthetic foot 3 is pivotably mounted around the ankle joint axis as the second pivot axis 4. The pivotable connection of lower leg tube and prosthetic foot forms the ankle joint 5. The orthopedic device 100 therefore has two upper parts 2 and two lower parts 3, wherein the lower leg part, as the connection between the two pivot axes 4, can be formed in one part or multiple parts and depending on the way it is considered is once the lower part and once the upper part.

The articulated connection of upper part 2 and lower part 3 around the respective pivot axis 4 forms the respective joint 5. In the illustrated exemplary embodiment, a resistance unit 9 in the form of an adjustable damper is arranged between the upper part 2 and the lower part 3 of the knee joint. The resistance unit 9 is supported with a proximal attachment device on the upper part 2 and with a distal attachment device on the lower part 3. The resistance unit 9 is designed in the exemplary embodiment as a passive component and influences a pivot movement of the upper part 2 relative to the lower part 3 around the pivot axis 4 both in the flexion direction and in the extension direction by converting kinetic energy into thermal energy. An actuator 6 for adjusting the respective resistance is assigned to the resistance unit 9. The actuator 6 acts on the resistance unit 9 according to the action principle. If the resistance unit 9 is designed, for example, as a pneumatic or hydraulic damper unit, the actuator 6 changes the flow cross section of the line from an extension chamber into the flexion chamber and back, in order to thus enlarge and shrink the respective flow cross section of an overflow channel. The flow resistance is thus reduced or increased. Alternatively or additionally to a change of the flow cross section, the actuator 6 can be designed as an adjustable magnet, for example, as an electromagnet which acts on a magnetorheological liquid. By changing the magnetic field, the viscosity of the magnetorheological liquid changes, so that the pivot resistance is changed via the change of the viscosity. The resistance unit 9 can also be designed as an electric motor which can be operated in generator operation, in which the flexion resistance and/or the extension resistance is changed by a corresponding generator regulation. In this case, the generator is generally the actuator. If a solely mechanical brake, such as a friction brake, is provided in which brake linings are pressed against a moving component, the actuator is the motor or drive using which the brake linings are pressed against the component.

Alternatively or additionally to a solely passive design of the resistance unit, the actuator 6 can also be designed as an active element, for example as an electric motor, in order not only to influence, but also actively induce, a relative movement of the upper part 2 in relation to the lower part 3. Alternatively to a design as an electric motor, the actuator 6 can also use other drive units or principles to release stored energy.

The actuator 6 is activated, deactivated, or modulated via a control unit 7. The flexion and/or extension is influenced and possibly blocked depending on the signal from the control unit. The movement behavior of the respective joint 5 during walking, standing, or another use is set by the control unit 7 using the corresponding signal. Sensors 8, which are arranged on the entire orthopedic device 100, are assigned to the control unit 7. The sensors 8 supply corresponding data wirelessly or via wired connections to the control unit 7. The data of the sensors 8 can be preprocessed and/or processed in the control unit 7 itself. Processors, memories, and all other necessary components are present in the control unit 7 or coupled thereto in order to evaluate the sensor data and, on the basis of this evaluation, perform a corresponding activation, deactivation, or modeling of the actuator and thus the resistance unit 9.

The control unit 7 in particular also has a storage unit 10 and can be coupled with a transmitter 11 and a receiver 12 in order to transmit sensor data, programs, access rights, settings, changes of settings, updates, or other things to external components or to components inside the orthopedic device. The sensors 8 detect all relevant parameters during the use of the orthopedic device, such as forces, torques, accelerations, temperatures, times, orientations in space, deformations, movement periods of time, usage periods of time, distances, relative movements, interactions with the surroundings, voltages, currents, biosignals, electromagnetic radiation, and the like. In particular, the sensors 8 or sensor units are designed as components which detect an angular position of the components in relation to one another and/or a spatial position or an orientation in space. In addition, the sensors 8 are designed to detect axial forces FA and torques MA. The forces and torques are determined everywhere where the detection is expedient and necessary, even if these forces and torques are shown only in conjunction with the ankle joint. Not all sensors 8 can detect all parameters; the arrangement and design of the sensors is directed according to the parameters to be detected in each case.

Derived variables can also be calculated from sensor values. For example, lever arms for certain points and/or force engagement points can be calculated from force and/or torque components, sensor values can be fused to form characteristic variables, for example, in IMUs (inertial measurement units), forces can be back-calculated from deformations, and/or a position can be back-calculated from multiple distances by triangulation. Such calculated variables are also included in the described embodiments and can be used for the control of the orthopedic device, in particular for the control of movement sequences having a pivot in the frontal plane.

In the exemplary embodiment, an electric motor is arranged on the ankle joint as the actuator 6, via which a resistance unit is provided via the generator operation and an assistance or an active displacement of the prosthetic foot relative to the lower leg part around the pivot axis 4 is provided in the motor operation as needed.

FIG. 2 shows an orthopedic device 100 as an orthosis of the lower extremity in an applied state. In this case, this is a KAFO (Knee Ankle Foot Orthosis), in which a first upper part 2 in the form of a thigh rail is secured via fastening devices 15 in the form of belts on a thigh. A first lower part 3 in the form of a lower leg rail is also arranged via fastening devices 15 on a lower leg of a user. The thigh rail and the lower leg rail are fastened pivotably on one another around a pivot axis 4 to form an orthotic knee joint 5. The components and technical units explained in FIG. 1, such as actuator, resistance unit, control unit, interfaces, and the like, are arranged on or in the orthotic knee joint 5. The sensors 8 are schematically shown. The second pivot axis 4 in the area of the natural ankle joint connects the lower leg rail as the second upper part 2 to a foot part as the second lower part 3. The unit for influencing the prosthetic ankle joint with respect to the resistance in the direction of the plantar flexion or the dorsiflexion is housed in the area of the orthotic ankle joint. Passive resistance units and/or active drives or actuators can also be provided here.

Both in the design as a prosthesis and in the design as an orthosis, in multiple joints 5 and corresponding resistance units, the actuators 6 for influencing the pivot movement around the respective pivot axis 4 can be controlled by a common control unit 7. It is also possible that multiple control units 7 are designed or arranged to control the orthopedic device 100 accordingly.

FIG. 3 schematically shows a first movement sequence of a person having an orthopedic device 100 in the form of a prosthetic leg, similar to that in FIG. 1. The person using the orthopedic device 100 stands essentially straight and upright. In the position shown, the left leg from the viewpoint of the user is the treated leg. A forward movement would be a movement out of the plane of the page. If the person using it now wishes to carry out a lateral movement from their position to the left, initially the left treated leg having the orthopedic device 100 is raised and moved laterally outward and/or an abduction movement is carried out, which is shown in the left illustration in the situation. After the prosthetic foot of the orthopedic device 100 is put down, the person using the latter displaces the weight to the treated side and pulls their right, untreated foot next to the prosthetic foot. The movement predominantly takes place in the frontal plane, which corresponds to the plane of the page. After the raising of the prosthetic foot, for example, a flexion movement within the knee is facilitated or initiated in that the flexion resistance is reduced or an active flexion assistance is initiated by the actuator (not shown). This movement is recognized, for example, by monitoring of the axial force curve within the prosthesis lower part or the prosthetic foot in conjunction with the monitoring of a movement and/or position of the orthopedic device in the frontal plane. If a lateral abducting movement is executed and the prosthetic foot is displaced laterally outward, even without an inclination or movement taking place within the sagittal plane or forward, a flexion resistance is reduced. Alternatively or additionally, an extension movement within the prosthetic knee joint is furthermore delayed or suppressed, so that a step to the side is possible. As soon as an axial load or a placement of the foot takes place, in one embodiment the flexion resistance is either increased or an extension is caused.

In the case of a solely lateral pivot or inclination, it is possible to suppress a plantar flexion and also to cause a dorsiflexion, so that the prosthetic foot can be placed over the full surface or with a straight sole essentially parallel to the underlying surface. Alternatively, the movement from position A to position B can be connected in an active foot to a plantar flexion, so that after the placement or during the placement, initially a tip of the foot is placed and a dorsiflexion takes place with increasing load. It is also possible that the foot is held in a position pointing slightly downward or is moved into this position during the movement from a position A to position B.

After the load, which is shown in position B, the flexion resistance is kept at a high level and/or an extension is carried out. In an active prosthesis having drives, initially a knee flexion is initiated. The knee angle can be kept constant in a specific position during the abduction until the hip is stretched. It is possible that at least one degree of freedom is kinematically coupled with at least one joint angle and/or one segment angle which can be sufficiently actuated by the person using the device. A kinematic coupling can be, among other things, a holonomic or non-holonomic constraint. Alternatively or additionally, it is possible that multiple degrees of freedom follow a kinematic coupling among one another. Alternatively or additionally to a kinematic coupling, multiple degrees of freedom can be subject to a force and/or torque coupling, by which a harmonic control of multiple degrees of freedom can be achieved.

FIG. 4 shows an alternative situation, in which initially the treated side having the orthopedic device 100 remains loaded and the healthy, untreated leg is displaced to the side. After the healthy leg is set down, the treated side is in a state inclined in the frontal plane within the standing phase. The inclination within the frontal plane is detected, possibly also an inclination in the sagittal plane. From a specific angle of inclination, in an active prosthesis, a knee flexion is first initiated. Then, depending on a time factor or the adduction or the pulling of the treated leg toward the untreated side or toward the body center, an extension or stretching takes place within the prosthetic knee joint.

FIG. 5 shows a movement pattern in which a user of an orthopedic device 100 wishes to walk around a curve, in the illustrated exemplary embodiment a left-hand curve. In this case, the treated leg having the orthopedic device 100 remains set down and is in the standing phase. The untreated leg is guided up to the frontal plane F and at the same time pivoted to the left. In a conventional prosthesis or orthosis from the prior art, it would not cause a release of a joint, in particular a reduction of a flexion resistance of the knee joint, in such a situation, since only a minor forward rotation within the orthopedic device 100 is detectable or the force engagement point on the foot remains in the middle of the foot. A flexion within the knee joint would not be triggered. However, according to the invention, a lateral rotation and an inclination within the frontal plane F of the treated side is detected. In the lateral step according to FIG. 5, which approximately corresponds to the situation in FIG. 4, an inclination from the prosthetic foot in the medial direction is detected, possibly in conjunction with a forward rotation from a position inclined to the rear of the lower leg part and/or the prosthesis socket. Upon a detection of a minor forward rotation in combination with a lateral rotation and an inclined position within the frontal plane F, the flexion resistance is changed, in particular reduced, so that the leg having the orthopedic device can easily be raised, flexed, and subsequently put down again.

For the detection of a lateral movement or an intended lateral movement, it is possible to carry out a force measurement within the distal end component, for example, in a footplate of an orthosis or in a prosthetic foot. The force introduction point travels during a movement within the frontal plane from the inside to the outside or from the outside to the inside in the ankle and not to the front and rear, so that an inference can be made from the course of the force introduction point within the orthopedic device 100 about which movement is currently being executed or will be executed. Alternatively or additionally, the orientation and/or tilting of the force vector and thus its change over time can be used to detect a lateral movement, in particular a tilt in the frontal plane. It is also possible to measure torques in the frontal plane and to conclude a pivot on the basis of an increase or decrease.

In the control of the resistance units or the movements in the joints of the orthopedic device 100 of the lower extremity, in addition to the movement sequences within the sagittal plane, those movement sequences and states or orientations are detected which take place in the frontal plane. The required sensor signals, which result in conclusions of a movement within the frontal plane and the type of the movement within the frontal plane, are derived via the sensors 8, in particular via an IMU. In addition to inclinations and pivots within the frontal plane, translational movements in specific planes within the frontal plane can be determined via a path integration of accelerations. Accelerations can be measured via an IMU. If the accelerations are not determined in an inertial-fixed coordinate system, in general the additional determination of the spatial position and a transformation into an inertial-fixed coordinate system are necessary for the integration.

FIG. 5 shows as an exemplary embodiment the control of a leg prosthesis or an orthosis during a direction change with an external rotation on the treated standing leg. The rotation of the treated leg takes place essentially in the hip in this case. The prosthetic foot or the foot part of the orthosis does not rotate or only rotates to a negligible extent in relation to the underlying surface in the standing phase. In principle, an internal rotation using the treated leg would also be possible. However, internal rotations are rather atypical and result in disadvantageous movement patterns in particular in patients having prosthetic legs. In contrast to walking forward, the treated leg does not roll forward in the standing phase, in particular at the end of the standing phase, within the sagittal plane around the ankle joint, but rather within the frontal plane toward the side. It is desirable here for the knee joint to enable an initiation of the swinging phase with a low bending resistance at the end of the standing phase upon such a movement or for a flexion movement to be initiated or at least assisted in the case of an active prosthetic knee joint.

It is therefore provided according to the invention that a swinging phase initiation is enabled or initiated with a reduction of the flexion resistance when the leg abducts in the frontal plane. In addition to a lateral movement, thus a pivot in the exemplary embodiment according to FIGS. 4 and 5, the lateral inclination as such can also be decisive. The inclination is used here in the sense of a tilt in relation to the vertical, which can also be independent of the direction of the tilt. Additionally, it can be required that the prosthetic knee joint is not located behind the pivot axis of the ankle joint, thus the leg is not tilted to the rear or no backward movement takes place. The change of the inclination is in particular not to take place within the posterior hemisphere, but rather if possible in the anterior hemisphere or during a movement in the forward direction.

FIG. 6 shows the sequence of a lifting movement of a treated leg, in the illustrated exemplary embodiment of a prosthetic leg. In principle, the statements for a prosthetic leg also apply for an orthotic treatment. From a stretched position, in which the prosthetic leg 100 stands on the ground, a hip band is carried out, due to which the prosthetic knee joint is flexed and the prosthetic foot is raised. The ankle joint is lifted essentially vertically upward. A plantar flexion takes place in the first lifting phase so that the tip of the foot falls downward. At the end of the lifting movement, the tip of the foot is free in the air in a plantar flexed position and is subsequently lifted by an actuator, so that a dorsiflexion takes place. Vice versa, to put it down, for example, during or after a lateral movement, first the tip of the foot is lowered or a plantar flexion is initiated or enabled, for example, by reducing the flexion resistance.

After the tip of the foot is put down at the desired position, a dorsiflexion is carried out by a load of the treated leg. Depending on the respective established inclination in the frontal plane and possibly in conjunction with further sensor values, corresponding actuators are activated or deactivated or target values for the actuator are modulated in order to make the putting down simple and enable it safely.

FIG. 7 shows a lateral movement similar to FIG. 4. Initially the untreated leg is lifted and put down in the frontal plane laterally of the prosthetic foot. In order to leave the body center of gravity as much as possible on a level, which is shown by the dashed line, in the course of the lateral movement upon a pivoting of the treated leg in the frontal plane, a plantar flexion is initiated, for example, by a drive or actuator, which results in a lengthening of the effective leg length. The lengthening is necessary so that a pivot movement can take place around the placement point of the prosthetic foot. The lengthening of the effective leg length by the plantar flexion is indicated by the prosthetic foot, which is shown lengthened. If the treated side is then pulled toward the other foot again, the prosthetic foot has to be lifted for this purpose. The unloading of the prosthetic foot can be detected, for example, via a contact switch or an axial force sensor. To facilitate the adduction movement, a dorsiflexion takes place, which can be reversed from a specific angle of inclination of the treated leg within the frontal plane, which results in a plantar flexion in order to enable the prosthetic foot to be put down softly.

FIG. 8 shows the reverse movement, namely the shifting of the treated side away from the untreated side. For this purpose, initially the treated side is lifted and moved within the frontal plane away from the standing foot. After the lifting, the prosthetic foot is still in the starting position without plantar flexion, and the knee joint is flexed. An effective leg length L results, which is advantageously maintained during the lateral movement. This is carried out by an extension Ext in the knee joint and a plantar flexion PF in the ankle joint, so that the sole of the foot and the hip pivot point essentially each remain on one level during the lateral movement, which is indicated by the dashed lines.

FIG. 9 shows a possible displacement of the prosthetic foot during a lateral angling of the prosthetic leg. During the spreading movement of the leg, a supination or a pronation of the prosthetic foot can take place, in order to be able to perform an adaptation of the leg length and an adaptation to the respective movement situation and the course of movement. FIG. 10 shows stepping over an obstacle, wherein the prosthetic foot can be put down on the same level as that of the standing leg. Alternatively, the putting down takes place on a higher or a lower level. Depending on the angular position of the treated leg, an activation of an actuator is carried out in order to be able to adapt the position of the prosthetic foot to the respective placement level.

FIG. 11 shows different movement patterns for a lateral movement within the frontal plane. In the upper row of movements, the movements are executed from the treated side and identified by capital letters, and in the lower row, the treated side is the standing leg, and the untreated side is moved, which is identified by lowercase letters. In principle, the movements can also be carried out in reverse. During the movement A, with a prosthetic foot standing at the rear, for example at the end of a step, in which the untreated side is in front, the treated side is moved both forward and also laterally up to the height of the untreated side so that a curve movement results. During the movement B, a movement reversal of the forward movement takes place, so that the treated side, for example, the prosthetic foot, is placed diagonally behind the untreated foot. During the movement C, the treated side is guided linearly in the sagittal plane up to the height of the untreated foot and then put down diagonally forward. In all three movements, a pivot of the treated side takes place within the frontal plane around a pivot point in the hip joint.

In the movements D to G, the starting position is a treated side with a prosthetic foot diagonally behind the untreated side. The treated side is guided in the movement D laterally adjacent to the untreated foot in a circular path movement, and during the movement E in front of the untreated foot in the scope of a cross step. During the movement F, the treated foot is moved over and placed diagonally in front of the untreated foot. During the movement G, a linear movement takes place from a spread-legged stance forward. In all movements, the treated side is located in the starting position in a position inclined in the frontal plane due to the spread-legged stance, during the movements E and F, the direction of inclination reverses, and, in the position D, the treated side is oriented vertically at the end of the movement.

The lateral movements away from the untreated side are shown in Figures I to J, wherein during the movement H, the movement takes place completely in the frontal plane, and, during the movements I and J, a movement takes place diagonally forward or diagonally to the rear. During the movements L and K, prosthetic feet standing in front of or behind the untreated foot are put down in the frontal plane at the height of the untreated foot. Both feet are located within the frontal plane.

The same or corresponding movements are executed in the lower row using the untreated side, and the prosthetic foot or the foot part of the treated side is the standing component here. The movements are executed accordingly and can be executed in both directions, thus instead of pulling in a movement away and vice versa. Inclinations in the frontal plane during the movements with the untreated side in the swinging phase take place via a rotation in the ankle joint with a fixed position or via a rotation around a foot placement point or a COP.

The initiation of the swinging phase can take place, with correspondingly stronger lateral inclination within the frontal plane or with a corresponding rotation or pivot around a distal pivot point, with less forward inclination or with less movement in the forward direction, wherein the respective inclinations and also the pivot speeds within the sagittal plane and the frontal plane can be weighted differently.

In addition to inclinations, pivot speeds, and inclination speeds as well as directions, it is possible for forces and torques on the orthopedic devices to be taken into consideration in the influencing of the joint movements.

If a corresponding movement is executed after the initiation of a swinging phase, when the treated side no longer has ground contact, this movement can be changed depending on the angles reached within the frontal plane, possibly in conjunction with the determined rotation values and load values of the scopes and courses of movement within the swinging phase. For example, a greater achievable flexion angle or a greater flexion can be permitted if a strong inclination is present and a correspondingly tight left-hand curve or right-hand curve is to be walked along.

In addition to influencing the flexion and extension of the prosthetic knee joint or orthotic knee joint of the orthopedic device 100 by activating, deactivating, or modulating the actuator, a prosthetic foot or an orthotic joint can also be influenced accordingly. For example, in the standing phase, upon a detection of an inclination or pivot within the frontal plane, a rolling over characteristic similar to that of walking can be activated, thus permitting a dorsiflexion or assisting a planter flexion, even if no forward rotation takes place. With an active prosthetic foot, an active plantar flexion can be initiated if a direction change is recognized via the lateral inclination within the frontal plane.

During a direction change as described above, it is possible to slow, restrict, or actively counteract a flexion of the knee joint and/or a dorsal extension of a foot in the standing phase. The movement momentum can thus be redirected better into the new walking direction. During these so-called braking steps, in which the prosthetic device is loaded by the body weight, an unrestricted flexion of the knee joint is permitted in the controls, which exclusively consider the sagittal movement. Upon a recognized lateral rotation, pivot, and/or inclination, in contrast, the flexion resistance is increased to prevent a further flexion. In an active prosthetic knee joint, an extension movement can be initiated. A prosthetic foot or an orthotic foot will provide a high resistance with respect to a dorsiflexion, so that the tip of the foot is not lifted. For this purpose, the corresponding resistances of a resistance unit are set via the actuator, and if necessary an active plantar flexion is executed with active units.

In solely passive orthopedic devices, no movements are initiated or assisted, but rather only resistances of different levels are provided in the respective movement directions around the respective pivot axes. Displacements in relation to one another are thus limited and the scope of movement or range of motion is restricted or movement speeds are modulated. In an ankle joint, the resistance against a dorsiflexion in the standing phase of lateral walking should not be reduced, in contrast to walking straight ahead, in particular in the middle standing phase. The initiation of a swinging phase is also problematic in passive prosthetic knee joints, since during lateral steps a resulting initiation of the hip movement is only minor and therefore only little knee flexion can be generated. When walking straight ahead, a knee flexion takes place due to the distal mass below the prosthetic knee joint and swinging up takes place due to the axial unloading and flexion. If the knee joint is flexed in the standing phase or flexion is caused in the unloaded phase, this bent position can be maintained during the pulling in or the abduction. This can be carried out with an increase of an extension resistance. Maintaining the flexed position is expedient if it is an unloaded lateral movement as shown, for example, in FIG. 4 in position D and in FIG. 3 in position A. A knee extension is advantageously released again in the course of the swinging phase, in particular released or controlled in a timely manner so that the leg can be put down again in the stretched position. This can take place, for example, on the basis of a recognition of the lateral movement, a recognition of braking of a lateral movement, a movement reversal, a thigh movement, and/or in a time-controlled manner.

While in prostheses the entire control of the movement via the actuator has to take place on the basis of the sensor data and the evaluation in the control unit, such a control at least partially also takes place via the muscular residual functionality in the case of an orthotic treatment, for example, a KAFO. The treated leg can often be lifted against gravity by the intrinsic musculature. Alternatively or additionally, the knee can be bent and/or the foot can be lifted. If a lateral step is then recognized at the end of a terminal standing phase or this becomes recognizable, for example, due to the reduction of the rotation speed or a corresponding loading curve, it is possible to reduce the resistances, in particular flexion resistances, in the knee joint and thus enable the user to move the orthosis with a minimal expenditure of force. If an orthotic treatment also includes the hips and the hip joint, a hip flexion and extension is also releasable during a lateral movement.

In active orthopedic devices 100, the degrees of freedom can be actively actuated during lateral steps. A prosthetic knee joint or an orthotic knee joint can be flexed in the unloading phase and the swinging phase, thus with decreasing axial load or no longer existing axial load, in order to create a sufficient ground clearance and to reduce compensation movements by plantar flexion of the contralateral side, also called “vaulting”. The knee angle can be set or limited in the swinging phase so that the distance between the hip and the foot shortens by a defined or relative absolute value. The distance between the hip joint and ankle joint is the so-called leg chord, which can also be used to control a lateral step upon an inclination within the frontal plane. The length of the leg chord is an essential control variable. The ground clearance can also be influenced by raising the tip of the foot or dorsiflexion, wherein keeping the foot in the neutral position can be sufficient. The orientation of the leg chord can also be used for the control, in particular the components thereof in the frontal plane.

The knee angle can be set or controlled so that the foot approximately remains below the hip joint, so that the knee bending angle increases when a stronger hip bend is carried out. The maximum bending angle is advantageously limited, so that the stretching movement can take place in a timely manner during a placement movement of the foot. The stretching movement within the knee angle or an end of an active flexion can take place in that the movement of the thigh is detected. If a hip extension is detected, an extension is facilitated or initiated.

If a corresponding drive or an activation is possible, a hip joint can be activated to achieve lifting of the foot.

With an actively driven foot, in the standing phase of a lateral movement, in which the body center of gravity is initially displaced from the treated, placed side to the contralateral side and the contralateral side is then placed, a plantar flexion can be executed in order to assist the lateral movement. In this way, the body center of gravity is prevented from sinking during a lateral movement and an inclination within the frontal plane, since the effective leg length of the treated side is increased. The transition between such a quasistatic length compensation and active pressing down is fluid and is dependent on the dynamics of the movement.

If the treated side having the orthopedic device is abducted in the swinging phase, thus moved in the lateral direction and set to the side, it can be advantageous to plantar flex the foot in the placement phase and enable controlled setting down on the forefoot. The functional vertical leg length or ground clearance is specifiable here as a target variable, which remains constant or is to be kept constant with increasing abduction. Alternatively or additionally, a transient course can be specified, in particular a chronologically specified course. If a step forward takes place at the same time, no plantar flexion is to occur, so that the forward movement is facilitated by a heel strike and rolling over the entire foot. During a backward step, a plantar flexion is advantageous.

Modern prosthetic devices have special modes, for example, for riding bicycles, sitting, rowing, or other special movements, in particular repetitive movements. If a lateral inclination or a lateral pivot is recognized, the recognition of a lateral inclination or sudden abduction can be used to switch off a corresponding special mode and activate a standard mode or safety mode in order to provide maximum safety for the user of the orthopedic device.

It is also possible to use a pivot in the frontal plane in situations for the control which do not correspond to a walking situation. For example, standing in a slightly or strongly straddled position can be recognized and the control can be adapted in relation to standing in a more closed foot position and/or vertical leg orientation. A foot having a degree of freedom in inversion-eversion degrees of freedom can be controlled, for example, so that the sole of the foot rests flatly on the ground, instead of on the inner edge.

A lateral inclination and/or movement can also be used in a special function. For example, the lateral inclination can be determined when skiing and the control can be adapted accordingly. In particular, it is possible to distinguish between uphill ski and downhill ski via the lateral inclination. With an inclination to the lateral, the corresponding side is the uphill ski, and with an inclination to the medial, it is the downhill ski. In order to distinguish between an inclination to medial and lateral, it is necessary to know whether it is a left or right leg. Such information can be stored in the orthopedic device or calculated from sensor values. If the treated side represents the downhill ski, for example, it is possible to suppress complete stretching of a knee joint, for example, by raising the movement resistance in the extension direction. More pressure can thus be exerted on the front part of the ski. If the treated side occupies the uphill ski side, the stretching can be completely permitted again. The change between uphill and downhill ski can be recognized via the tilt and the transition can possibly be made to be fluid. In addition to skiing, the control can also be adapted depending on the lateral inclination and/or movement in other special functions, in particular movement resistances and/or movements can be adapted.

An orthosis or an exoskeleton can permit a movement, counteract it, or assist a movement. In an exoskeleton, the person using it usually has no significant restrictions of the locomotor system. Movements are accordingly assisted, for example, to reduce the load of the body, increase performance, or enhance comfort. In an orthosis, an insufficiency of the person using it is usually compensated, for example, muscular or neuronal insufficiency is compensated by corresponding assistance. The assistance by an exoskeleton and/or an orthosis can be adapted during a lateral movement. For example, a movement can be permitted if a lateral movement and/or inclination is detected. It is also possible that a movement which is initiated by the person using it is facilitated by the orthosis or the exoskeleton, i.e., the required forces and torques which have to be applied by the body in order to carry out a movement are reduced. During a lateral step, for example, it is possible that the weight of the orthosis or the exoskeleton and/or the weight of one's own limbs are assumed. Forces and torques are thus applied which counteract gravity. In an unloading and/or lifting phase, a knee-bending and/or hip-flexing torque is applied to facilitate the angling of the leg. During the swinging phase, the leg can be kept in the angled position by a knee-bending and/or hip-flexing torque. The swinging of the leg to the side or the lateral positioning of the foot can be facilitated by a hip-abducting or hip-adducting torque. The flexing of the leg can be facilitated by a plantar flexing torque in an unloading phase. A dorsal-extending torque in the lifting phase, but also in the swinging phase during the positioning of the foot, can facilitate the lifting of the foot and thus achieve sufficient ground clearance in the swinging phase. In a placement phase, the hip-bending, knee-bending, and/or dorsal-extending torque can be reduced in order to enable stretching of the leg and controlled setting down of the foot. Alternatively or additionally, a hip-extending, knee-stretching, and/or plantar-flexing torque can be applied in order to actively assist the lowering. In the standing phase, a hip abduction torque and/or hip adduction torque can also be applied to assist the lateral walking.

During the use of an exoskeleton, it is possible to compensate for or reduce the weight and the inertial effects of the exoskeleton itself. By applying forces and torques which counteract these weight and/or inertial effects, the wearing comfort can be enhanced and the effort of the person using the exoskeleton for carrying out movements can be reduced. Ideally, the exoskeleton appears mechanically transparent from the viewpoint of the person using it, i.e., the additional weight and/or the inertia are not perceptible. For such a control it is possible to detect movements and movement changes in the frontal plane and also incorporate them. In particular a tilt in the frontal plane in relation to gravity or a potential field or also the rotational movement in the frontal plane can be used for the control. If, for example, in a knee-encompassing exoskeleton, an unloaded, angled leg, in which the foot is located below the hip, tilts laterally in relation to gravity, i.e., tilts in the frontal plane from the vertical in the direction of the horizontal, the knee-stretching torque is reduced due to the weight of the lower leg segment of the exoskeleton. If a knee-bending torque is applied in the vertical position by an actuator of the exoskeleton in order to counteract the knee-stretching effect of the lower leg weight, this torque can be reduced with increasing lateral inclination. This also applies for an orthosis.

1. A method for controlling an orthopedic device of a lower extremity, wherein the orthopedic device comprises an upper part and a lower part, wherein the upper part and the lower part are mounted on one another in an articulated manner around at least one pivot axis to form a joint, and wherein the orthopedic device comprises at least one actuator coupled to a control unit which activates or deactivates the at least one actuator based on sensor data of at least one sensor coupled with the control unit to influence a pivot resistance and/or a relative movement of the upper part (2) in relation to the lower part, comprising: (3), characterized in that an
detecting orientation and/or displacement of the orthopedic device in a frontal plane using sensor data of the at least one sensor; and
activating or deactivating the actuator or modulating a target value for the actuator based on an orientation and/or displacement of the orthopedic device in the frontal plane.

2. The method as claimed in claim 1, wherein the sensor data is detected, and the activating or deactivating of the actuator or the modulating of the target value occurs during use of the orthopedic device in the applied state.

3. The method as claimed in claim 1, wherein detecting the orientation and/or displacement of the orthopedic device is performed using a spatial position sensor, an inertial measurement unit (IMU), and/or angle sensors.

4. The method as claimed in claim 1 further comprising:
detecting forces, torques, and/or accelerations via sensors; and used
using detected forces, torques, and/or accelerations as a basis for control of activating or deactivating the actuator.

5. The method as claimed in claim 1 wherein the orthopedic device is designed as a prosthesis or orthosis and has an artificial knee joint and/or an artificial ankle joint, to which the actuator is assigned.

6. The method as claimed in claim 1, further comprising:
detecting translational displacements of the orthopedic device; and
using detected translational displacements as a basis for control of activating or deactivating the actuator.

7. The method as claimed in claim 1 further comprising reducing a flexion resistance or initiating a flexion upon reaching a threshold value of an inclination or pivot of a treated side in a standing phase in a medial direction.

8. The method as claimed in claim 7, further comprising reducing the flexion resistance or initiating the flexion upon a reduction of an axial load or in a lifting phase on the treated side.

9. The method as claimed in claim 7 wherein reducing the flexion resistance or initiating the flexion is only performed during a forward inclination or a forward pivot.

10. The method as claimed in claim 1 wherein, upon an inclination or pivot of a treated side in a standing phase in a medial direction with simultaneous detection of a backward inclination or a backward pivot, no reduction of a flexion resistance is performed or an increase of a flexion resistance is initiated.

11. The method as claimed in claim 1 wherein, upon an inclination or pivot of a treated side in a standing phase in a medial direction with simultaneous detection of a backward inclination or a backward pivot, a flexion movement is stopped or an extension movement is initiated.

12. The method as claimed in claim 1 further comprising adjusting a maximum pivot angle of the upper part in relation to the lower part depending on an inclination or pivot in the frontal plane.

13. The method as claimed in claim 1 wherein, upon an inclination or a pivot of a treated side in a standing phase in a medial direction, driving an artificial ankle joint in a plantar flexion direction or increasing or not reducing a dorsiflexion resistance.

14. The method as claimed in claim 1 wherein, upon an inclination or a pivot of a treated side in a standing phase and upon attaining an established flexion torque, no reduction of a flexion resistance is performed or initiating an increase of the flexion resistance is performed or an extension torque is applied.

15. The method as claimed in claim 1 wherein, upon a pivot of a treated side in a lifting phase of a swinging phase in a lateral direction, reducing a flexion resistance or initiating a flexion.

16. The method as claimed in claim 15, further comprising delaying or preventing an extension in an artificial knee joint in a swinging phase.

17. The method as claimed in claim 16, further comprising releasing the extension in the artificial knee joint or causing one or more of, in a time-controlled manner, after reaching a pivot angle or on a basis of a hip extension movement, a change of a pivot speed, and a change of a pivot direction.

18. The method as claimed in claim further comprising activating or deactivating a special mode of control based on an inclination or displacement in a frontal plane.

19. An orthopedic device of a lower extremity, comprising: having
an upper part;
a lower part, wherein the upper part and the lower part are mounted with one another in an articulated manner around at least one pivot axis to form a joint;
at least one actuator coupled or coupleable to a control unit which activates or deactivates the actuator based on sensor data of at least one sensor coupled or coupleable to the control unit in order to influence a pivot resistance and/or a relative movement of the upper part in relation to the lower part,
wherein the at least one sensor is designed and configured to detect sensor data about an orientation and/or displacement of the orthopedic device in a frontal plane.
wherein the control unit is configured to activate or deactivate the actuator, or wherein the control unit is configured to modulate a target value for the actuator based on an orientation and/or displacement in the frontal plane.

20. The orthopedic device as claimed in claim 19, wherein the at least one sensor is designed as an inertial measurement unit (IMU) and is fastened on the upper part or the lower part.

21. The orthopedic device as claimed in claim 19 further comprising at least one force sensor, acceleration sensor, angle sensor, and/or torque sensor arranged on the upper part and/or the lower part.
